(19) United States
(12) Patent Application Publication
(10) Pub. No.: US 2023/0293817 A1
(43) Pub. Date: Sep. 21, 2023

(54) PRECISION DOSE DELIVERY DEVICE PLUNGER ROD INSERTION GAUGE AND METHODS THEREFOR

(71) Applicant: REGENERON PHARMACEUTICALS, INC., Tarrytown, NY (US)

(72) Inventors: Jason SMITH, Latham, NY (US); Trevor LANGLEY, Rensselaer, NY (US); Nicholas MCDONNELL, Albany, NY (US)

(73) Assignee: REGENERON PHARMACEUTICALS, INC., Tarrytown, NY (US)

(21) Appl. No.: 18/187,564

(22) Filed: Mar. 21, 2023

Related U.S. Application Data
(60) Provisional application No. 63/321,901, filed on Mar. 21, 2022.
(60) Provisional application No. 63/342,515, filed on May 16, 2022.

(51) Int. Cl. A61M 5/178

(52) U.S. Cl. CPC A61M 5/178; A61M 2005/2006

(57) ABSTRACT

A plunger rod insertion gauge for a precision dose delivery device is disclosed. The insertion gauge includes: an insertion gauge body including a handle portion and a gauge portion; wherein the gauge portion includes one or more plunger rod abutting surfaces and one or more flange abutting surfaces oriented substantially perpendicular to a plunger rod insertion axis; and wherein a dimension measured between the one or more plunger rod abutting surfaces and the one or more flange abutting surfaces defines a maximum plunger rod insertion depth for the precision dose delivery device. Other aspects are described and claimed.

### CROSS REFERENCE TO RELATED APPLICATIONS

This application claims priority to U.S. Application No. 63/321,901, filed on Mar. 21, 2022, and U.S. Application No. 63/342,515, filed on May 16, 2022, both of which are incorporated by reference herein in their entireties.

### FIELD OF DISCLOSURE

Aspects of the present disclosure relate to devices and methods for assembling, priming, or otherwise configuring a dose delivery device, e.g., a syringe, to promote precision dose delivery. More specifically, embodiments of the present disclosure relate to devices for aiding in the insertion of the plunger of a precision dose delivery device.

### INTRODUCTION

Drug products including fluid drug substances may be deliverable to patients in a variety of ways, including via injection. In many cases, the precision and accuracy of a liquid drug product's volume is crucial. For example, medical professionals may have an interest in ensuring that an approved or prescribed volume of a drug substance is consistently delivered to each patient requiring the drug. Additionally, over- or under-dosing a patient with a drug substance, even slightly, may have an undesired (or even negative) clinical impact on the patient. Moreover, some drug products are prescribed at low volumes (e.g., under approximately 100 μL). At low volumes, human error in preparing and delivering an accurate dose of a drug substance for injection may impact the drug's efficacy in a patient and the subsequent clinical effect on the patient.

During the assembly of devices for providing these accurate doses of drug substances, a plunger will often have to be inserted into a device body to a proper depth. Exceeding this depth may cause stopper movement which can jeopardize the integrity of the seals of the drug delivery devices.

The entire disclosure of commonly owned U.S. Pat. No. 11,439,758, which discloses a dose delivery device, is incorporated by reference herein except for any definitions, subject matter disclaimers or disavowals, and except to the extent that the incorporated material is inconsistent with the express disclosure herein, in which case the language in this disclosure controls.

### SUMMARY

According to certain aspects of the disclosure, an insertion gauge device is disclosed that engages with components of a precision dose delivery device during assembly of the device.

In one aspect, a plunger rod insertion gauge for a precision dose delivery device is provided. The insertion gauge includes: an insertion gauge body including a handle portion and a gauge portion; wherein the gauge portion includes one or more plunger rod abutting surfaces and one or more flange abutting surfaces oriented substantially perpendicular to a plunger rod insertion axis; and wherein a dimension measured between the one or more plunger rod abutting surfaces and the one or more flange abutting surfaces defines a maximum plunger rod insertion depth for the precision dose delivery device.

In another aspect, a plunger rod insertion gauge for a precision dose delivery device is disclosed. The insertion gauge including: a gauge spacer comprising a first gauge spacer surface and a second gauge spacer surface; a receiving block positioned adjacent to the gauge spacer and comprising a first receiving block surface and a second receiving block surface; and a protruding element positioned above the receiving block; wherein a dimension between the first gauge spacer surface and the second gauge spacer surface defines a maximum plunger rod insertion depth for the precision dose delivery device.

In yet another aspect, a method of assembling a dose delivery device is disclosed. The method includes: inserting a lower end of a plunger rod axially into the device body through an opening in a flange portion of the dose delivery device; positioning an insertion gauge laterally adjacent to an upper end of the plunger rod; and applying an axial force to the insertion gauge to translate the plunger rod axially into the opening in the flange portion until the insertion gauge abuts the flange portion and the plunger rod.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated into and constitute a part of this specification, illustrate an embodiment of a device for aiding in the insertion of the plunger of a precision dose delivery device during for example, assembly, and, together with the description, serve to explain principles of the disclosed embodiment(s). The drawings show multiple aspects of the present disclosure.

FIG. 1 depicts an exemplary insertion gauge device and precision dose delivery device, according to some embodiments of the present disclosure.

FIG. 2 depicts a perspective view of an insertion gauge device, according to some embodiments of the present disclosure.

FIG. 3 depicts an exemplary insertion gauge device engaged with a precision dose delivery device, according to some embodiments of the present disclosure.

FIG. 4 depicts an exemplary insertion gauge device having a handle portion, according to some embodiments of the present disclosure.

FIG. 5 depicts a front view of the insertion gauge device of FIG. 4, according to some embodiments of the present disclosure.

FIG. 6 depicts a side view of the insertion gauge device of FIG. 4, according to some embodiments of the present disclosure.

FIG. 7 depicts a top view of the insertion gauge device of FIG. 4, according to some embodiments of the present disclosure.

FIG. 8 depicts an exemplary flowchart of a method of assembling a plunger rod into the body of a drug delivery device containing a predetermined amount of a liquid (e.g., a formulated drug substance) using an insertion gauge, according to one or more embodiments.

### DETAILED DESCRIPTION

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended to reflect or indicate that the embodiment(s) is/are one “example,” rather than “ideal.” In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items. Additionally, the terms “about,” “approximately,” “substantially,” and the like, when used in describing a numerical value, denote a variation of +/−10% of that value, unless specified otherwise.

Embodiments of the present disclosure may be used with any type of fluid-containing products, such as liquid drug substances, liquid placebos, or other liquids that may be dispensed in a dose form. As used herein, the term “drug substance” may refer to a formulated substance including an active ingredient or ingredients, such as, e.g., small or large molecules, such as pain medications, steroids, or biologics. As used herein, the term “biologic” may refer to a large molecule (e.g., having a size greater than 15 kDa, greater than 30 kDa, greater than 50 kDa, greater than 75 kDa, or greater than 100 kDa) created in a living system such as a cell. Biologics may include proteins (e.g., antibodies), nucleic acids, large sugars, etc. Unlike small molecules that may have well-defined chemical structures, biologics may have highly complex structures that cannot be easily quantified by laboratory methods. As used herein, the term “drug product” may refer to a volume of a drug substance apportioned into a primary packaging component for packaging, transportation, delivery, and/or administration to a patient.

The term “primary packaging component” refers to a packaging component for a drug product, such as a drug container, that is designed and manufactured to be in direct physical contact with the formulated drug substance. (See, for example, Guidance for Industry on Container Closure Systems for Packaging Human Drugs and Biologics, U.S. Department of Health and Human Services, Food and Drug Administration, Center for Drug Evaluation and Research, and Center for Biologics Evaluation and Research (May 1999), which is incorporated by reference herein.) Examples of primary packaging components include pre-fillable syringes, Luer syringes, cartridges, and vials made of glass, plastic, other polymers or co-polymers, and/or other materials.

As used herein, the terms “distal” and “distally” refer to a location (or portion of a device) relatively closer to, or in the direction of, a patient delivery site, and the terms “proximal” and “proximally” refer to a location (or portion of a device) relatively closer to, or in the direction of, a user end opposite a distal location/portion of a device.

As used herein, the term “body,” when used in reference to a part of a device, may refer to a component of the device suitable for containing a volume of a drug substance. A body may include, e.g., a barrel (such as a syringe barrel), tube, cylinder, or other containing portion of a device. For example, in some embodiments, a body may be a syringe barrel made of glass and/or a barrel that may be free of silicone oil, substantially free of silicone oil, and/or contain less than 50 μg of silicone oil. In some embodiments, a body may also include a distal end portion having a nozzle, needle, needle attachment site, and/or distal cap.

Embodiments of the present disclosure may be used with products typically having small dose volumes, such as, e.g., ophthalmic drug products. In some embodiments, devices of the present disclosure may be used with drug products including ophthalmic solutions having, for example, no more than 5 particles≥25 μm in diameter per ml and/or no more than 50 particles≥10 μm in diameter per ml. In some embodiments, devices of the present disclosure may be used with drug products including a large molecule, e.g., a molecular weight of 30 kDA or greater. In some embodiments, devices of the present disclosure may be used with drug products including a fragment of a large molecule. For example, in some embodiments, devices of the present disclosure may be used with drug products including an antigen-binding molecule. In some aspects, the antigen-binding molecule may be an antibody or antigen-binding fragment. In some embodiments, devices of the present disclosure may be suitable for use with vascular endothelial growth factor (VEGF) inhibitors and/or large molecule and small molecule antagonists of VEGF and/or angiopoietin-2. In some embodiments, devices of the present disclosure may be suitable for use with drug products including ingredients such as, e.g., aflibercept, alirocumab, abicipar pegol, bevacizumab, brolucizumab, conbercept, dupilumab, evolocumab, tocilizumab, certolizumab, abatacept, rituximab, infliximab, ranibizumab, sarilumab, adalimumab, anakinra, trastuzumab, pegfilgrastim, interferon beta-1a, insulin glargine [rDNA origin], epoetin alpha, darbepoetin, filigrastim, golimumab, etanercept, antigen-binding fragments of any of the above, or combinations of such binding domains, such as a bispecific antibody to VEGF or angiopoietin-2, among others. Drugs suitable for use with dose delivery devices according to the present disclosure may be used to treat, for example, an ocular disease such as choroidal neovascularization, wet age-related macular degeneration, macular edema secondary to retinal vein occlusion (RVO) (including branch RVO and central RVO), choroidal neovascularization secondary to pathologic myopia (PM), diabetic macular edema (DME), diabetic retinopathy, or proliferative retinopathy.

In some embodiments, devices and aspects of the present disclosure may aid in providing accurate dose delivery while also maintaining the container closure integrity (CCI). Further, preassembling the agent in the devices of the present disclosure may minimize the steps necessary for preparing a dose for delivery to a patient. During this assembly process, proper insertion of the plunger rod into the drug-delivery device body may prevent undesirable stopper movement, and may support precise and sterile drug dosing.

In some embodiments, devices according to the present disclosure may be used in the preparation of precision drug delivery devices. For example, in some embodiments, devices according to the present disclosure may engage a portion of a precision drug delivery device in order to mechanically prevent over-insertion of the plunger rod into the drug delivery device body.

Currently in the dose delivery device market, and specifically in the pre-filled syringe market, there is a need for mechanisms that allow a user to set precisely for delivery a small volume of a product in a syringe (e.g., a pre-filled or fillable/refillable syringe), while maintaining closed container integrity. Embodiments of the present disclosure may assist manufacturers, drug product providers, medical professionals, and/or patients with accurately making, filling, or otherwise preparing a dose administration device. Moreover, embodiments of the present disclosure may assist in preventing or mitigating errors or variation in device manufacture or use, such as errors in plunger rod insertion and stopper positioning.

Embodiments or aspects of embodiments disclosed herein may be used in conjunction with existing syringe body parts to modify off-the-shelf products, which may reduce the development and manufacturing time for the dose delivery devices. In other instances, embodiments or aspects of embodiments disclosed herein may be provided to users along with precision drug delivery devices during their manufacture. The syringes with which plunger rod insertion gauges described herein may be used may be pre-filled or may be fillable/refillable.

In some embodiments, precision drug delivery devices according to the present disclosure may be depicted as including one type of plunger rod and plunger, or as including a general schematic representation of a plunger rod and plunger. For example, some devices according to the present disclosure may be depicted or described as including, e.g., a plunger rod having a ball-tipped end, which engages with a stopper such that the plunger rod and the stopper may be attached together. In some embodiments, the plunger rod and stopper may not be physically secured to one another. In this manner, pushing the plunger rod distally may urge the stopper distally, but retracting the plunger rod proximally would not retract the stopper. It is contemplated that plunger rod insertion gauges may be designed, adapted, and/or configured for use with multiple and/or different configurations of plunger rods and stoppers as may be appropriate.

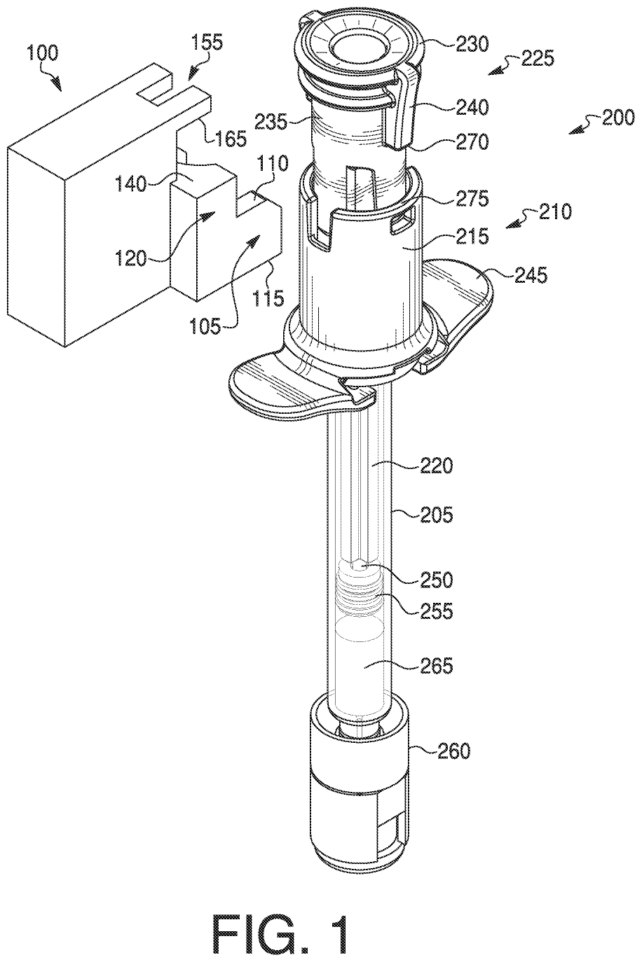

Referring now to FIG. 1, a plunger rod insertion gauge 100 according to an embodiment of the present disclosure is depicted adjacent to an exemplary precision drug delivery device 200. The drug delivery device 200 can include a body 205, and a blocking component in the form of a flange piece 210 with a proximal collar 215 surrounding an opening through which a plunger rod 220 may pass. The plunger rod 220 can include an actuation portion 225 in the form of a plunger head which may be actuated (e.g., pushed or twisted) to rotate the plunger rod 220, or to move plunger rod 220 longitudinally into the body 205. The actuation portion 225 may contain a first part 230 and a second part 235. The first part 230 may be torus-shaped whereas the second part 235 may be cylindrically-shaped. The first part 230 may be positioned longitudinally adjacent to the second part 235 and a diameter of the first part 230 may be greater than a diameter of the second part 235.

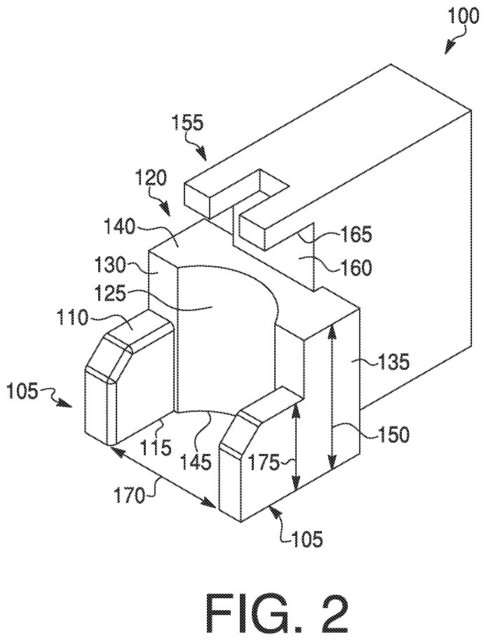

The flange piece 210 may be of any suitable size and/or shape to serve as a blocking component in the drug delivery device 200, to close, partially close, cover, or partially cover the end of body 205 opposite the end through which the drug is expelled and which is also covered by closure 260 (e.g., a tamper-evident closure). In some embodiments, the flange piece 210 can include a proximal collar 215 configured to engage with body 205 to hold the flange piece 210 in place in relation to the body 205. The proximal collar 215 of flange piece 210 may be sized and configured to accept part of actuation portion 225 of plunger rod 220, while blocking protrusions 240 of plunger rod 220 from moving distally past a predetermined point until plunger rod 220 is rotated to a particular position. As shown in FIGS. 1-2, the proximal collar 215 may be cylindrical; in alternate embodiments, the proximal collar 215 may have any suitable size or shape compatible with actuation portion 225. In some embodiments, flange piece 210 may include one or more flanges 245, which may be sized and configured to aid a user in holding drug delivery device 200 and/or expelling a formulated drug substance from drug delivery device 200.

The plunger rod 220 may be rotatable about a central longitudinal axis (e.g., in one direction or in both directions), for example, by grasping and/or twisting actuation portion 225 of the plunger rod 220 relative to the flange piece 210 and/or body 205. In some embodiments, protrusions 240 may assist a user in grasping and/or twisting actuation portion 225 relative to flange piece 210 and/or body 205, by providing additional surface area that a user may grasp and/or push against to twist actuation portion 225. The plunger rod 220 can include a distal tip 250 sized and configured to push, attach to, or otherwise interface with a stopper 255. The stopper 255 can form a portion of the seal between the substance 265 and the environment, an aspect of the CCI necessary to maintain drug safety and efficacy. Proper stopper positioning and suitable limitations on movements that may compromise the CCI may be beneficial in ensuring consistent filling and proper sealing.

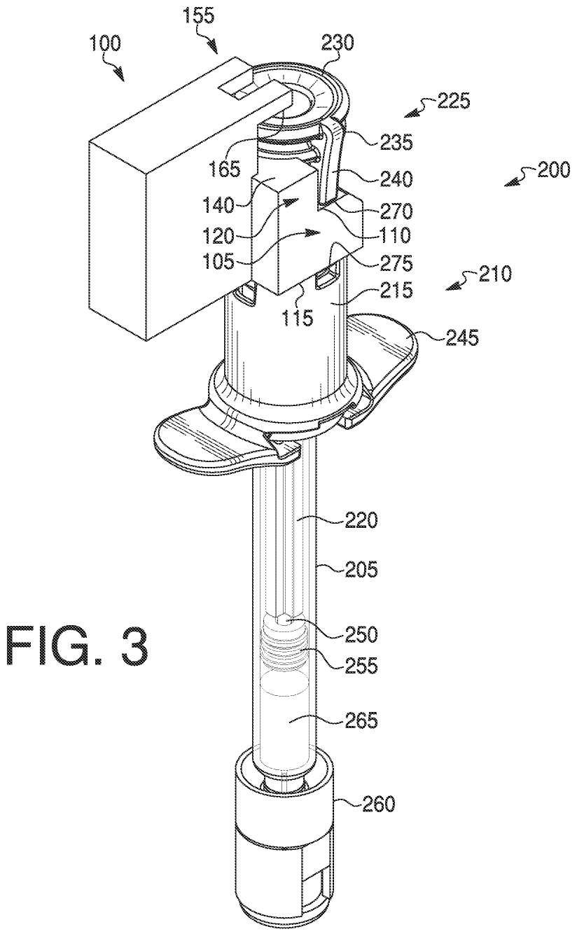

In accordance with the present disclosure, a plunger rod insertion gauge (“insertion gauge”) 100 may be associated with the precision drug delivery device 200 during the insertion of the plunger rod 220 to aid in proper rod insertion, which in turn can aid in proper stopper positioning and maintenance of the CCI. The plunger rod insertion gauge 100 may be shaped to cooperate with one or more surfaces of the flange piece 210, proximal collar 215, and/or actuation portion 225 of the plunger rod 220 such that, when engaged (as shown in FIG. 3), the plunger rod 220 is prevented from being inserted into the precision drug delivery device body 200 more than a desired amount, thereby defining a maximum plunger rod insertion depth of the plunger rod 220.

FIG. 2 depicts an exemplary plunger rod insertion gauge 100, in accordance with the present disclosure. The insertion gauge 100 may include gauge spacers 105 positioned substantially opposite one another. Each of the gauge spacers 105 of the insertion gauge 100 may include a first surface 110 and a second surface 115. The second surface 115 may be located opposite the first surface 110 and both the first surface 110 and the second surface 115 may be substantially flat.

The insertion gauge 100 may also include a receiving block 120 positioned adjacent to the gauge spacers 105. The receiving block 120 may contain a receiving wall 125, a first and second vertical surface 130, 135, and a first and second horizontal surface 140, 145. The receiving wall 125 may have an arcuate shape, complimentary to the second part 235 of the actuation portion 225 of the drug delivery device 200. A height of each of the first and second vertical surfaces 130, 135 may be defined by the distance 150 between the first and second horizontal surfaces 140, 145.

In some embodiments, the second horizontal surface 145 may be the same as the second surface 115 of the gauge spacer 105 or may otherwise extend in the same plane as the second surface 115 of the gauge spacer 105. In some embodiments, the first and second vertical surfaces 130, 135 may be flush with an outer surface of the gauge spacer 105.

The insertion gauge 100 may also include a protruding element 155 that may extend into a space in a direction away from the second receiving wall 160 between the projections of gauge spacers 105 and over the first horizontal surface 140 and the receiving wall 125. A second receiving wall 160 may be positioned between the first horizontal surface 140 of the receiving block 120 and a first protruding surface 165 of the protruding element 155.

The combination of the gauge spacers 105 form a receiving area for the actuation portion 225 of the plunger rod 220. More particularly, a space 170 between the gauge spacers 105 may be larger than the diameter of the second part 235 of the actuation portion 225 of the plunger rod 220 so that when the insertion gauge 100 interacts with the drug delivery device 200, the second part 235 of the actuation portion 225 may fit between the gauge spacers 105 and register/contact with the receiving wall 125 of the receiving block 120 of the insertion gauge 100. Upon registration, the first surface 110 of each gauge spacer 105 may interact with a bottom surface 270 of a protrusion 240 and the second surface 115 of each gauge spacer 105 may interact with a top surface 275 of the proximal collar 215, as further illustrated and described herein. Additionally, upon registration, the first part 230 of the actuation portion 225 may fit within the space between the first horizontal surface 140 and the first protruding surface 165 of the protruding element 155. In some embodiments, the first part 230 of the actuation portion 225 may contact the second receiving wall 160.

The height 175 of the gauge spacer 105 may serve as the critical dimension of the plunger rod insertion gauge 100 that dictates the depth to which the plunger rod 220 may be inserted into the body 205 of the drug delivery device 200. Accordingly, the height of the gauge spacer 105 may be inversely related to the insertion depth of the plunger 220. More particularly, a higher height of the gauge spacer 105 may correspond to a shallower insertion depth of the plunger rod 220, and vice versa.

The height 175 of the gauge spacers 105 may vary between insertion gauges depending on the maximum plunger rod insertion depth needed. For instance, insertion gauge A having a first height may be employed in a situation that dictates that a first volume of a drug solution needs to be deployed whereas insertion gauge B having a second, higher height may be employed in a situation that dictates that a second, lower volume of a drug solution needs to be deployed. The particular profiling of the insertion gauge 100 (i.e., the various contours and/or dimensions of the insertion gauge 100) may be generic to multiple drug delivery devices or can be customized to match more closely to a particular plunger rod design. For instance, the surface features of an actuation component of a plunger rod for Drug Delivery Device A may be different than the surface features of an actuation component of a plunger rod for Drug Delivery Device B. In such a situation, two separate plunger rod insertion gauges may be employed, wherein a profile of each of these insertion gauges is shaped to conform to the surface features of their respective drug delivery device. To support insertion gauge reuse, the insertion gauge may be made from a material such as a plastic and/or rubber that is both compatible with assembly environment (i.e., capable of sterilization, resistance to any solvents used), and non-marring to the drug delivery device elements in order to avoid marking or damage to the drug delivery device.

FIG. 3 depicts the plunger rod insertion gauge 100 interacting with the actuation portion 225 of the plunger rod 220 to define a position of the plunger rod 220. Once the plunger rod 220 is inserted to the desired depth (as determined by the insertion gauge 100 employed), one or more surfaces of the plunger rod insertion gauge 100 will interfere with one or more surfaces of the flange piece 210 (e.g., such as the top surface 275 of the proximal collar 215) and/or actuation portion 225 (e.g., such as the bottom surface 270 of the protrusion 240 of the actuation portion 225). At this time, the plunger rod 220 and/or stopper 255 will be prevented from further longitudinal movement into the device body 205, thereby defining a final expulsion volume of the formulated drug substance 265, and the plunger rod insertion gauge 100 may be disengaged. In some embodiments, the insertion gauge 100 is not fixedly secured (e.g., is not frictionally attached (e.g., via snap fit) to the actuation portion 225 of the plunger rod 220 to the plunger rod 220, but rather, may be free to translate independently from the plunger rod 220, along the second part 235 of the plunger rod 220, at least in a direction perpendicular to the longitudinal axis of the plunger rod 220. In other embodiments, the insertion gauge 100 may be selectively attached to the plunger rod 220. As a result, the plunger rod insertion gauge 100 may be reusable for subsequent filling of the same or different drug delivery devices.

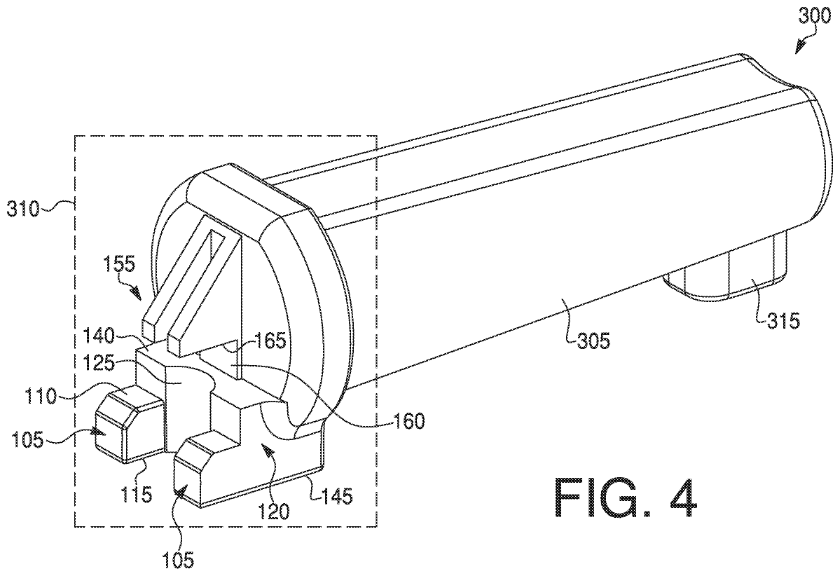

FIG. 4 depicts an exemplary handled plunger rod insertion gauge 300 containing an extended portion configured for use as a handle. The handle portion 305 may extend from a surface of the insertion gauge portion 310 that is opposite the side configured to abut a plunger rod precision drug delivery device 200. The handle portion 305 can be shaped to allow a user to securely grasp the gauge 300, which can aid in the positioning of the insertion gauge portion 310 against a plunger rod, applying an axial force to the insertion gauge portion 310, and separating the insertion gauge portion 310 from the actuation portion 225 of the plunger rod. In some embodiments the size and shape of the handle portion 305 may be altered so as to better fit the hand of a user. As depicted in FIG. 4, a downward protrusion 315 may be included at the end of the handle portion 305 to prevent the insertion gauge portion 310 from slipping from the hand of a user. Other features that may be included to better fit the hand of a user can include, for example, adding one or more of: finger indentations, surface finishes for improved grip, and/or a through-hole through which a finger or strap may pass.

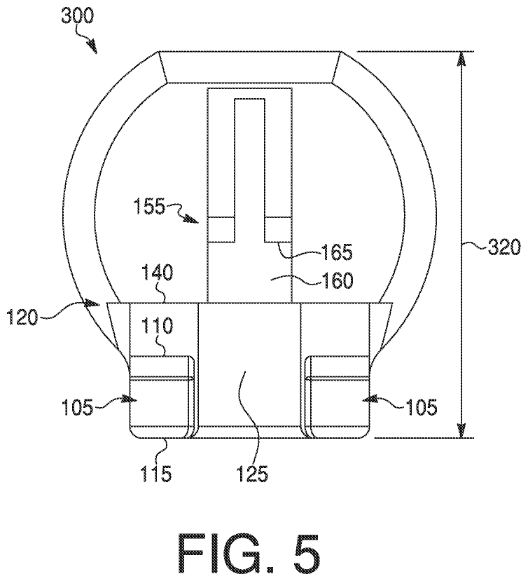

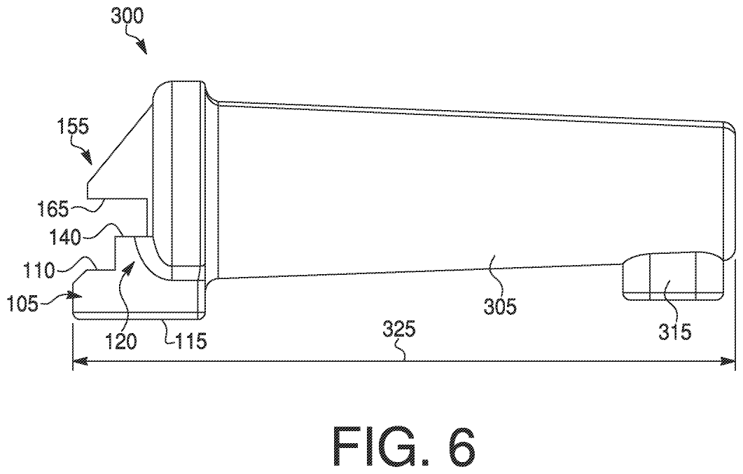

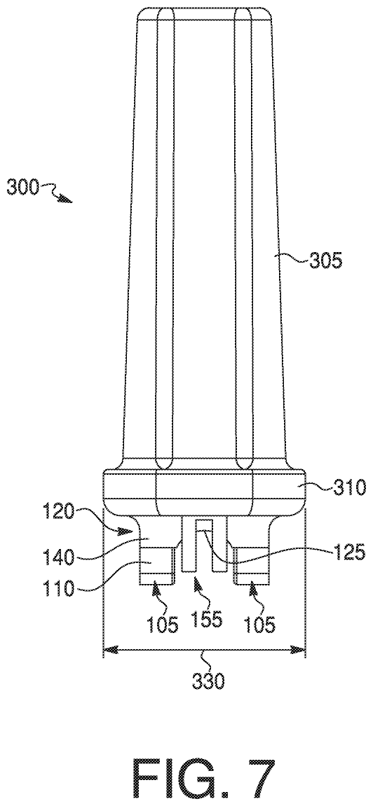

Referring now collectively to FIGS. 5-7, a variety of different views of the handled insertion gauge 300 illustrated in FIG. 4 are provided, in accordance with the present disclosure. FIG. 5 depicts a front view of the handled insertion gauge 300. From this perspective, the height 320 of the handled insertion gauge 300 may be perceived. Although the height 320 may be virtually any height, in some embodiments, the height may be about 30 mm to about 40 mm, about 30 mm, about 31 mm, about 32 mm, about 33 mm, about 34 mm, about 35 mm, about 36 mm, about 37 mm, about 38 mm, about 39 mm, about 40 mm. In one embodiment, the height may be 35.93 mm. FIG. 6 depicts a side view of the handled insertion gauge 300. From this perspective, the length 325 of the handled insertion gauge 300 may be perceived. Although the length 325 may be virtually any length, in some embodiments, the length may be about 80 mm to about 120 mm, about 90 mm to about 100 mm. In one embodiment, the length may be 100 mm. FIG. 7 depicts a top view of the handled insertion gauge 300. From this perspective, the maximum width 330 of the insertion gauge portion 310 of the handled insertion gauge 300 may be perceived. Although the maximum width 330 may be virtually any width, in some embodiments, the width may be about 30 mm to about 40 mm, about 30 mm, about 31 mm, about 32 mm, about 33 mm, about 34 mm, about 35 mm, about 36 mm, about 37 mm, about 38 mm, about 39 mm, about 40 mm. In one embodiment, the maximum width may be 35 mm.

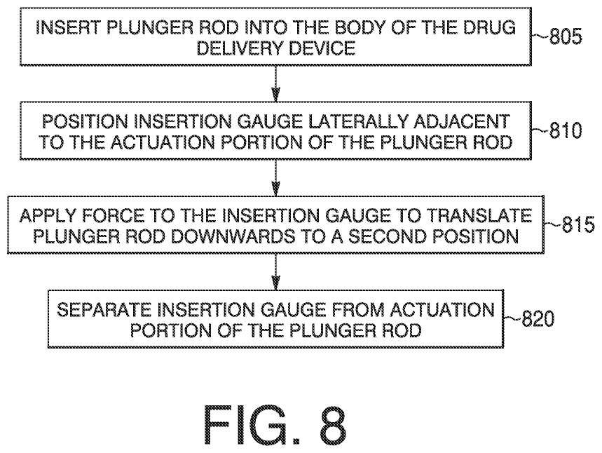

FIG. 8 depicts a flowchart illustrating an exemplary method 800 of assembling a plunger rod into the body of a drug delivery device containing a predetermined amount of a liquid (e.g., a formulated drug substance) using an insertion gauge, according to one or more embodiments of the present disclosure.

At step 805, a plunger rod 220 of a drug delivery device 200 may be inserted into the device body 205 containing a liquid (e.g., a formulated drug substance 265). The insertion gauge 100 or 300 may optionally be positioned laterally adjacent to the actuation portion 225 of the plunger rod 220 during this step.

At step 810, an insertion gauge 100 or 300 may be positioned laterally adjacent to the actuation portion 225 of the plunger rod 220. In this regard, a second part 235 of the actuation portion 225 may fit through a space 170 defined by the gauge spacer(s) 105 and may contact a receiving wall 125 of the insertion gauge 100 or 300. Simultaneously, a first part 230 of the actuation portion 225 may fit between a space provided by a first horizontal surface 140 of a receiving block 120 and a first protruding surface 165 of a protruding element 155. In the contacted state, the first surface 110 of each gauge spacer 105 may contact a bottom surface 270 of a protrusion 240 of the actuation portion 225.

At step 815, an axial force may be applied to the insertion gauge 100 to translate the plunger rod 220 downwards to a second position at which a second surface 115 of the gauge spacer(s) 105 abuts a top surface 275 of a proximal collar 215 of a flange piece 210 of the drug delivery device 200. The force application may cause the first protruding surface 165 of the protruding element 155 to contact a top surface of the first part 230 of the actuation portion 225, thereby causing the plunger rod 220 to move downward into the body 205 of the drug delivery device 200. Liquid in the drug delivery device 200 at step 805 may be expelled from the drug delivery device 200 until contact between the second surface 115 of the gauge spacer 105 and the top surface 275 of the proximal collar 215 is achieved. The remaining liquid in the drug delivery device 200 subsequent to application of the force may define a dose volume.

At step 820, the insertion gauge 100 or 300 may be separated from the actuation portion 225 of the plunger rod 220. More particularly, the insertion gauge 100 may be translated laterally in a direction substantially perpendicular to the direction of the applied force in step 815.

Components of the devices described herein may be designed and/or suited for manufacture in one or more ways. In some embodiments, for example, the plunger rod insertion gauge may be suitable for manufacture via, e.g., injection molding, 3-dimensional printing, or machining. Precision may be particularly important, for example, to ensure proper fitment and range of motion of the plunger rod during insertion.

Embodiments of the present disclosure may include the following features:

Item 1. A plunger rod insertion gauge for a precision dose delivery device, the insertion gauge comprising: an insertion gauge body including a handle portion and a gauge portion; wherein the gauge portion includes one or more plunger rod abutting surfaces and one or more flange abutting surfaces oriented substantially perpendicular to a plunger rod insertion axis; and wherein a dimension measured between the one or more plunger rod abutting surfaces and the one or more flange abutting surfaces defines a maximum plunger rod insertion depth for the precision dose delivery device.

Item 2. The insertion gauge of Item 1, wherein the one or more plunger rod abutting surfaces include at least one surface configured to abut a portion of a plunger rod located between a top surface of the plunger rod and a top surface of a flange portion of the precision dose delivery device.

Item 3. The insertion gauge of Item 2, wherein the one or more plunger rod abutting surfaces include at least one surface configured to abut the top surface of the plunger rod.

Item 4. The insertion gauge of Item 2, wherein the portion of the plunger rod is a protruding element and wherein the one or more plunger rod abutting surfaces include at least one surface configured to abut a bottom surface of the protruding element.

Item 5. The insertion gauge of Item 2, wherein the one or more plunger rod abutting surfaces include a first receiving surface and a second receiving surface and wherein the portion of the plunger rod includes a first actuation portion and a second actuation portion, wherein the first actuation portion is torus-shaped and wherein the second actuation portion is cylindrically-shaped.

Item 6. The insertion gauge of Item 5, wherein the first receiving surface is configured to abut the second actuation portion of the plunger rod and wherein the second receiving surface is configured to abut the first actuation portion of the plunger rod, wherein the first receiving surface is positioned below the second receiving surface.

Item 7. The insertion gauge of Item 2, wherein the top surface of the flange portion is the top surface of a proximal collar of the flange portion.

Item 8. The insertion gauge of Item 1, wherein the handle portion includes one or more features to aid a user in grasping the insertion gauge.

Item 9. A plunger rod insertion gauge for a precision dose delivery device, the insertion gauge comprising: a gauge spacer comprising a first gauge spacer surface and a second gauge spacer surface; a receiving block positioned adjacent to the gauge spacer and comprising a first receiving block surface and a second receiving block surface; and a protruding element positioned above the receiving block; wherein a dimension between the first gauge spacer surface and the second gauge spacer surface defines a maximum plunger rod insertion depth for the precision dose delivery device.

Item 10. The insertion gauge of Item 9, wherein the receiving block further comprises a first receiving surface and wherein the first receiving surface is arcuate-shaped.

Item 11. The insertion gauge of Item 10, wherein the first receiving surface is configured to abut a first part of an actuation portion of a plunger rod of the precision dose delivery device.

Item 12. The insertion gauge of Item 9, further comprising a receiving area defined between the first receiving block surface of the receiving block and a first surface of the protruding element.

Item 13. The insertion gauge of Item 12, wherein a second part of an actuation portion of a plunger rod of the precision dose delivery device is configured to reside in the receiving area.

Item 14. The insertion gauge of Item 9, wherein the first gauge spacer surface is configured to abut a portion of a plunger rod of the precision dose delivery device and wherein the second gauge spacer surface is configured to abut a portion of a flange component of the precision dose delivery device.

Item 15. A method of assembling a dose delivery device, the method comprising: inserting a lower end of a plunger rod axially into the device body through an opening in a flange portion of the dose delivery device; positioning an insertion gauge laterally adjacent to an upper end of the plunger rod; and applying an axial force to the insertion gauge to translate the plunger rod axially into the opening in the flange portion until the insertion gauge abuts the flange portion and the plunger rod.

Item 16. The method of Item 15, wherein the insertion gauge comprises an insertion gauge body including a handle portion and a gauge portion.

Item 17. The method of Item 16, wherein the handle portion includes one or more features to aid a user in grasping the insertion gauge during the positioning.

Item 18. The method of Item 16, wherein the positioning comprises positioning one or more plunger rod abutting surfaces of the gauge portion against one or more surfaces of the upper end of the plunger rod.

Item 19. The method of Item 15, wherein a maximum insertion depth of the plunger rod is defined when the insertion gauge abuts the flange portion and the plunger rod.

Item 20. The method of Item 15, further comprising, after the insertion gauge abuts the flange portion and the plunger rod, separating the insertion gauge from the upper end of the plunger rod by translating the insertion gauge laterally, in a direction substantially perpendicular to the direction of the axial force.

The many features and advantages of the present disclosure are apparent from the detailed specification, and thus, it is intended by the appended claims to cover all such features and advantages of the present disclosure that fall within the true spirit and scope of the disclosure. Further, since numerous modifications and variations will readily occur to those skilled in the art, it is not desired to limit the present disclosure to the exact construction and operation illustrated and described, and accordingly, all suitable modifications and equivalents may be resorted to, falling within the scope of the present disclosure.

Devices of the present disclosure are operable to assist in providing consistent and accurate plunger rod insertion for drug delivery devices. Those skilled in the art will appreciate that the conception upon which this disclosure is based may readily be used as a basis for designing other devices, methods, and systems for carrying out the purposes of the present disclosure. Accordingly, the claims are not to be considered as limited by the foregoing description.

We claim:

1. A plunger rod insertion gauge for a precision dose delivery device, the insertion gauge comprising:
an insertion gauge body including a handle portion and a gauge portion;
wherein the gauge portion includes one or more plunger rod abutting surfaces and one or more flange abutting surfaces oriented substantially perpendicular to a plunger rod insertion axis; and
wherein a dimension measured between the one or more plunger rod abutting surfaces and the one or more flange abutting surfaces defines a maximum plunger rod insertion depth for the precision dose delivery device.

2. The insertion gauge of claim 1, wherein the one or more plunger rod abutting surfaces include at least one surface configured to abut a portion of a plunger rod located between a top surface of the plunger rod and a top surface of a flange portion of the precision dose delivery device.

3. The insertion gauge of claim 2, wherein the one or more plunger rod abutting surfaces include at least one surface configured to abut the top surface of the plunger rod.

4. The insertion gauge of claim 2, wherein the portion of the plunger rod is a protruding element and wherein the one or more plunger rod abutting surfaces include at least one surface configured to abut a bottom surface of the protruding element.

5. The insertion gauge of claim 2, wherein the one or more plunger rod abutting surfaces include a first receiving surface and a second receiving surface and wherein the portion of the plunger rod includes a first actuation portion and a second actuation portion, wherein the first actuation portion is torus-shaped and wherein the second actuation portion is cylindrically-shaped.

6. The insertion gauge of claim 5, wherein the first receiving surface is configured to abut the second actuation portion of the plunger rod and wherein the second receiving surface is configured to abut the first actuation portion of the plunger rod, wherein the first receiving surface is positioned below the second receiving surface.

7. The insertion gauge of claim 2, wherein the top surface of the flange portion is the top surface of a proximal collar of the flange portion.

8. The insertion gauge of claim 1, wherein the handle portion includes one or more features to aid a user in grasping the insertion gauge.

9. A plunger rod insertion gauge for a precision dose delivery device, the insertion gauge comprising:
a gauge spacer comprising a first gauge spacer surface and a second gauge spacer surface;
a receiving block positioned adjacent to the gauge spacer and comprising a first receiving block surface and a second receiving block surface; and
a protruding element positioned above the receiving block;
wherein a dimension between the first gauge spacer surface and the second gauge spacer surface defines a maximum plunger rod insertion depth for the precision dose delivery device.

10. The insertion gauge of claim 9, wherein the receiving block further comprises a first receiving surface and wherein the first receiving surface is arcuate-shaped.

11. The insertion gauge of claim 10, wherein the first receiving surface is configured to abut a first part of an actuation portion of a plunger rod of the precision dose delivery device.

12. The insertion gauge of claim 9, further comprising a receiving area defined between the first receiving block surface of the receiving block and a first surface of the protruding element.

13. The insertion gauge of claim 12, wherein a second part of an actuation portion of a plunger rod of the precision dose delivery device is configured to reside in the receiving area.

14. The insertion gauge of claim 9, wherein the first gauge spacer surface is configured to abut a portion of a plunger rod of the precision dose delivery device and wherein the second gauge spacer surface is configured to abut a portion of a flange component of the precision dose delivery device.

15. A method of assembling a dose delivery device, the method comprising:
inserting a lower end of a plunger rod axially into the device body through an opening in a flange portion of the dose delivery device;
positioning an insertion gauge laterally adjacent to an upper end of the plunger rod; and
applying an axial force to the insertion gauge to translate the plunger rod axially into the opening in the flange portion until the insertion gauge abuts the flange portion and the plunger rod.

16. The method of claim 15, wherein the insertion gauge comprises an insertion gauge body including a handle portion and a gauge portion.

17. The method of claim 16, wherein the handle portion includes one or more features to aid a user in grasping the insertion gauge during the positioning.

18. The method of claim 16, wherein the positioning comprises positioning one or more plunger rod abutting surfaces of the gauge portion against one or more surfaces of the upper end of the plunger rod.

19. The method of claim 15, wherein a maximum insertion depth of the plunger rod is defined when the insertion gauge abuts the flange portion and the plunger rod.

20. The method of claim 15, further comprising, after the insertion gauge abuts the flange portion and the plunger rod, separating the insertion gauge from the upper end of the plunger rod by translating the insertion gauge laterally, in a direction substantially perpendicular to the direction of the axial force.
